(19) United States
(12) Patent Application Publication
(10) Pub. No.: US 2024/0132436 A1
(43) Pub. Date: Apr. 25, 2024

(54) ESTER-BASED COMPOUND HAVING LOW TRACTION COEFFICIENT, LUBE BASE OIL COMPRISING SAME COMPOUND, AND LUBRICANT COMPOSITION COMPRISING SAME COMPOUND

(71) Applicants: SK INNOVATION CO., LTD., Seoul (KR); SK ENMOVE CO., LTD., Seoul (KR)

(72) Inventors: Jun Soo SON, Daejeon (KR); Nam Kyu BANG, Daejeon (KR); Seung Eon LEE, Daejeon (KR); Ji Su JEONG, Daejeon (KR)

(21) Appl. No.: 18/446,490

(22) Filed: Aug. 9, 2023

(30) Foreign Application Priority Data
Sep. 30, 2022 (KR) 10-2022-0125252

(51) Int. Cl. C07C 53/134; C10M 105/24

(52) U.S. Cl. CPC C07C 53/134; C10M 105/24; C10M 2207/126

(57) ABSTRACT

Proposed is a compound represented by Formula 1, where R₁ is an alkyl group having 9 to 30 carbon atoms, an alkenyl group having 9 to 30 carbon atoms, or an alkynyl group having 9 to 30 carbon atoms, R₂ is hydrogen, an alkyl group having 1 to 10 carbon atoms, an alkenyl group having 2 to 10 carbon atoms, or an alkynyl group having 2 to 10 carbon atoms, and R₃ to R₅ are each independently hydrogen, an alkyl group having 1 to 10 carbon atoms, an alkenyl group having 2 to 10 carbon atoms, or an alkynyl group having 2 to 10 carbon atoms. Alternatively, at least two of R₃ to R₅ are bonded together to form a hydrocarbon ring having 5 or 6 carbon atoms. In addition, proposed are a lube base oil and a lubricant composition comprising the compound.

### CROSS REFERENCE TO RELATED APPLICATION

The present application claims priority to Korean Patent Application No. 10-2022-125252, filed Sep. 30, 2022, the entire contents of which is incorporated herein for all purposes by this reference.

### BACKGROUND OF THE DISCLOSURE

### 1. Field of the Disclosure

The present disclosure relates to a novel ester-based compound, a lube base oil comprising the ester-based compound, and a lubricant composition comprising the ester-based compound.

### 2. Description of the Related Art

A lube base oil is a raw material for a lubricating oil product. Typically, good lube base oils have high viscosity index (VI), excellent stability (such as oxidation stability, heat stability, UV stability, etc.), and low volatility. The American Petroleum Institute (API) classifies lube base oils according to qualities, as shown in Table 1 below.

TABLE 1

| Classification | Sulfur (%) | Saturate (%) | Viscosity index (VI) |
|---|---|---|---|
| Group I | >0.03 | <90 | 80 ≤ VI < 120 |
| Group II | ≤0.03 | ≥90 | 80 ≤ VI < 120 |
| Group III | ≤0.03 | ≥90 | 120 ≤ VI |

| | |
|---|---|
| Group IV | All polyalphaolefins (PAOs) |
| Group V | All other lube base oils not included in |
| | Group I, II, III, or IV |

In vehicles, increasing fuel efficiency or mileage is highly important for improving energy efficiency. To this end, the development of lube base oils and/or lubricants with excellent friction reduction performance is essential to minimize friction-induced energy loss in engines or motors. In addition, biodegradable lube base oils and/or lubricants are more likely to be easily degradable by microorganisms than existing lube base oils and/or lubricants, thereby minimizing negative impacts on the environment. Thus, the use of biodegradable lube base oils and/or lubricants with excellent friction reduction performance is desirable in terms of improving energy efficiency and eco-friendliness as well as reducing hazards to the human body.

### SUMMARY OF THE INVENTION

The present disclosure provides a novel ester-based compound having a low traction coefficient, a lube base oil comprising the same compound, and a lubricant composition comprising the same compound.

A first aspect of the present disclosure provides a compound represented by Formula 1.

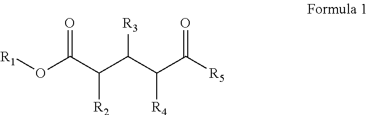

Here, R₁ is an alkyl group having 9 to 30 carbon atoms, an alkenyl group having 9 to 30 carbon atoms, or an alkynyl group having 9 to 30 carbon atoms, R₂ is hydrogen, an alkyl group having 1 to 10 carbon atoms, an alkenyl group having 2 to 10 carbon atoms, or an alkynyl group having 2 to 10 carbon atoms, and R₃ to R₅ are each independently hydrogen, an alkyl group having 1 to 10 carbon atoms, an alkenyl group having 2 to 10 carbon atoms, or an alkynyl group having 2 to 10 carbon atoms. Alternatively, at least two of R₃ to R₅ are bonded together to form a hydrocarbon ring having 5 or 6 carbon atoms.

According to an embodiment, R₁ is an alkyl group having 9 to 30 carbon atoms, R₂ is hydrogen or an alkyl group having 1 to 10 carbon atoms, and at least two of R₃ to R₅ are bonded together to form a hydrocarbon ring having 5 or 6 carbon atoms.

According to an embodiment, the compound is represented by Formula 2, when R₃ and R₅ in the compound of Formula 1 are bonded together to form a hydrocarbon ring having 5 carbon atoms.

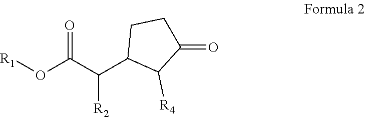

Here, R₁ is an alkyl group having 9 to 30 carbon atoms, an alkenyl group having 9 to 30 carbon atoms, or an alkynyl group having 9 to 30 carbon atoms, R₂ is hydrogen, an alkyl group having 1 to 10 carbon atoms, an alkenyl group having 2 to 10 carbon atoms, or an alkynyl group having 2 to 10 carbon atoms, and R₄ is hydrogen, an alkyl group having 1 to 10 carbon atoms, an alkenyl group having 2 to 10 carbon atoms, or an alkynyl group having 2 to 10 carbon atoms.

According to an embodiment, R₁ is an alkyl group having 9 to 30 carbon atoms, R₂ is hydrogen or an alkyl group having 1 to 10 carbon atoms, and R₄ is hydrogen or an alkyl group having 1 to 10 carbon atoms.

A second aspect of the present disclosure provides a lube base oil, wherein the lube base oil comprises the compound according to the first aspect of the present.

According to an embodiment, the lube base oil has a traction coefficient of 0.04 or less when being measured under conditions of a load of 38 N, a temperature of 100° C., a speed of 1000 mm/s, and a slide-to-roll ratio (SRR) of 100%.

According to an embodiment, the lube base oil has a flash point of 200° C. or higher.

According to an embodiment, the lube base oil comprises at least 50 wt % of the compound based on the total weight of the lube base oil.

A third aspect of the present disclosure provides a lubricant composition, wherein the lubricant composition comprises the compound according to the first aspect of the present disclosure.

According to an embodiment, the lubricant composition comprises greater than 0 wt % and less than 50 wt % of the compound based on the total weight of the lubricant composition.

The present disclosure provides a lube base oil and/or a lubricant composition that comprise a novel ester-based compound having a low traction coefficient. The lube base oil and/or the lubricant composition of the present disclosure can provide excellent friction reduction performance as well as biodegradability.

### DESCRIPTION OF THE EMBODIMENTS

Even though the above and other objectives, features, and advantages of the present disclosure will be more clearly understood from the following detailed description taken in conjunction with the accompanying drawings, the present disclosure is not necessarily limited thereto. In addition, when it is determined that the detailed description of the known art related to the present disclosure might obscure the gist of the present disclosure, the detailed description thereof will be omitted.

### Compound

The present disclosure provides a novel compound. The compound may be represented by Formula 1.

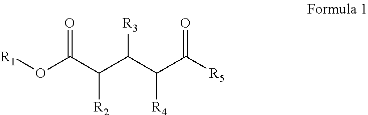

Here, R₁ is an alkyl group having 9 to 30 carbon atoms, an alkenyl group having 9 to 30 carbon atoms, or an alkynyl group having 9 to 30 carbon atoms, R₂ is hydrogen, an alkyl group having 1 to 10 atoms, an alkenyl group having 2 to 10 atoms, or an alkynyl group having 2 to 10 carbon atoms, and R₃ to R₅ are each independently hydrogen, an alkyl group having 1 to 10 carbon atoms, an alkenyl group having 2 to 10 carbon atoms, or an alkynyl group having 2 to 10 carbon atoms. Alternatively, at least two of R₃ to R₅ are bonded together to form a hydrocarbon ring having 5 or 6 carbon atoms.

The alkenyl group provided herein may have at least one double bond. In addition, the alkynyl group provided herein may have at least one triple bond.

Furthermore, it should be understood that unless otherwise specified, the alkyl group, the alkenyl group, and the alkynyl group provided herein include a branched-chain or straight-chain alkyl group, alkenyl group, or alkynyl group.

Moreover, it should be understood that unless otherwise specified, the alkyl group, the alkenyl group, and the alkynyl group provided herein include a substituted or unsubstituted alkyl group, alkenyl group, or alkynyl group. In the present disclosure, the “substituted” refers to replacing at least one carbon or hydrogen atom constituting each group with another substance. In an embodiment, the alkyl group, the alkenyl group, or the alkynyl group in the compound is an unsubstituted alkyl group, alkenyl group, or alkynyl group, respectively.

In an embodiment of the present disclosure, R₁ in the compound of Formula 1 may be an alkyl group having 9 to 30 carbon atoms, an alkenyl group having 9 to 30 carbon atoms, or an alkynyl group having 9 to 30 carbon atoms. In an embodiment, R₁ may be the alkyl group having 9 to 30 carbon atoms. In an embodiment, R₁ may be the anan alkyl group having 9 to 20 carbon atoms. In an embodiment, R₁ may be the alkyl group having 9 to 16 carbon atoms.

In the present disclosure, the number of carbon atoms in R₁ is 9 or more. It has been found, that when the number of carbon atoms in R₁ is less than 9, there may be a problem in that the traction coefficient and flash point to be described later are unachievable. In addition, due to a low viscosity index, viscosity changes significantly with temperature variation, so lubrication performance is likely to be poorly exhibited. On the other hand, when the number of carbon atoms in R₁ exceeds 30, the compound is highly likely to be present in a solid state at temperatures of 0° C. or higher due to a high pour point. Thus, it is problematic in that the compound is difficult to be used as a component constituting a lube base oil or lubricant composition.

As described above, R₂ in the compound of Formula 1 may be hydrogen, an alkyl group having 1 to 10 carbon atoms, an alkenyl group having 2 to 10 carbon atoms, or an alkynyl group having 2 to 10 carbon atoms. In an embodiment, R₂ may be hydrogen or an alkyl group having 1 to 10 carbon atoms. In an embodiment, R₂ may be hydrogen in terms of providing further excellent friction reduction performance due to low steric hindrance around an ester group capable of being adsorbed onto the metal surface. On the other hand, when the number of carbon atoms in R₂ exceeds 10, the viscosity is excessively increased, so it is problematic in that the compound is difficult to be used as a component constituting the lube base oil or the lubricant composition.

In addition, as described above, R₃ to R₅ in the compound of Formula 1 may be each independently hydrogen, an alkyl group having 1 to 10 carbon atoms, an alkenyl group having 2 to 10 carbon atoms, or an alkynyl group having 2 to 10 carbon atoms. For example, R₃ to R₅ may be different from each other or the same. Alternatively, only the two of R₃ to R₅ may be the same.

Alternatively, at least two of R₃ to R₅ may be bonded together to form the hydrocarbon ring having 5 or 6 carbon atoms. For example, R₃ and R₄, R₃ and R₅, R₄ and R₅, or R₃, R₄, and R₅ may be bonded together to form a hydrocarbon ring.

In an embodiment of the present disclosure, R₃ and R₅ in the compound of Formula 1 may be bonded together to form the hydrocarbon ring having 5 or 6 carbon atoms. In an embodiment, R₃ and R₅ are bonded together to form the hydrocarbon ring having 5 carbon atoms. Furthermore, the compound may be represented by a compound represented by Formula 2.

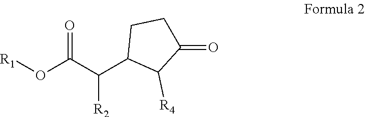

Here, R₁ and R₂ may be the same as R₁ and R₂ in the compound of Formula 1 described above.

In the compound of Formula 2, R₄ may be hydrogen, the alkyl group having 1 to 10 carbon atoms, the alkenyl group having 2 to 10 carbon atoms, or the alkynyl group having 2 to 10 carbon atoms. In an embodiment, R₄ may be hydrogen or the alkyl group having 1 to 10 carbon atoms. In an embodiment, R₄ may be the alkyl group having 1 to 10 carbon atoms. In an embodiment, R₄ may be the alkyl group having 3 to 7 carbon atoms. In an embodiment, R₄ may be the alkyl group having 5 carbon atoms. When the number of carbon atoms in R₄ exceeds 10, the viscosity is excessively increased, so it is problematic in that the compound is difficult to be used as a component constituting the lube base oil or the lubricant composition.

The compound of the present disclosure may be a product of an esterification reaction between an organic acid and an alcohol, where the organic acid has a carboxyl group. Without wishing to be bound by theory, the compounds of the present disclosure may be biodegradable due to the ester bond present in the compounds.

For example, the compound may be a product of an esterification reaction between jasmonic acid (C₁₂H₁₈O₃) and an alcohol. In an embodiment of the present disclosure, the jasmonic acid may be dihydrojasmonic acid also known as 2-(3-oxo-2-pentylcyclopentyl)acetic acid and having the molecular formula C₁₂H₂₀O₃.

In an embodiment, the alcohol may be an alcohol having 9 to 30 carbon atoms. In an embodiment, the alcohol may be an alcohol having 9 to 20 carbon atoms. In an embodiment, the alcohol may be an alcohol having 9 to 16 carbon atoms.

The compound of the present disclosure may constitute at least a portion of the lube base oil or the lubricant composition, as described above. The compound can improve the friction reduction performance as well as the biodegradation performance of the lube base oil and the lubricant composition.

### Lube Base Oil

The present disclosure provides a lube base oil comprising the compound of the first aspect described above. In an embodiment of the present disclosure, the lube base oil may comprise the compound in an amount of at least about 50 wt % based on the total weight of the lube base oil. For example, the lube base oil may comprise the compound in an amount in a range of about 50 wt % to about 100 wt %, such as about 55 wt % to about 100 wt %, about 60 wt % to about 100 wt %, about 70 wt % to about 100 wt %, about 80 wt % to about 100 wt %, about 90 wt % to about 100 wt %, about 95 wt % to about 100 wt %, about 50 wt % to about 90 wt %, about 60 wt % to about 90 wt %, about 70 wt % to about 90 wt %, about 80 wt % to about 90 wt %, about 50 wt % to about 80 wt %, about 60 wt % to about 80 wt % %, about 70 wt % to about 80 wt %, about 50 wt % to about 70 wt %, and about 60 wt % to about 70 wt %.

The lube base oil of the present disclosure may have a low traction coefficient. In the related art, as lubricating oil products or the like have a lower traction coefficient, performance capable of reducing the friction of mechanical equipment is considered to be better, thereby increasing the energy efficiency of the mechanical equipment.

In an embodiment of the present disclosure, the lube base oil may have a traction coefficient of about 0.04 or less when being measured under conditions: a load of 38 N, a temperature of 100° C., a speed of 1000 mm/s, and a slide-to-roll ratio (SRR) of 100%. For example, the traction coefficient may be about 0.037 or less, and about 0.035 or less. When the traction coefficient exceeds 0.04, there may be a problem in that the excellent friction reduction performance to be achieved in the present disclosure is unrealizable.

In an embodiment of the present disclosure, the lube base oil may have a flash point of about 200° C. or higher. For example, the lube base oil may have a flash point in a range of about 200° C. to 300° C., such as about 200° C. to 210° C., about 200° C. to 220° C., about 200° C. to 230° C., about 200° C. to 240° C., about 200° C. to 250° C., about 200° C. to 260° C., about 200° C. to 270° C., about 200° C. to 280° C., and about 200° C. to 290° C. When the flash point is lower than 200° C., the lube base oil is highly ignitable and should not be used as a lubricating oil product.

The lube base oil of the present disclosure may have a kinematic viscosity within a predetermined range. In an embodiment of the present disclosure, the lube base oil may have a kinematic viscosity at a temperature of 100° C. in a range of about 2 cSt to 10 cSt, such as about 2 cSt to 3 cSt, about 2 cSt to 4 cSt, about 2 cSt to 5 cSt, about 2 cSt to 6 cSt, about 2 cSt to 7 cSt, about 2 cSt to 8 cSt, about 2 cSt to 9 cSt, about 3 cSt to 4 cSt, about 3 cSt to 5 cSt, about 3 cSt to 6 cSt, about 3 cSt to 7 cSt, about 3 cSt to 8 cSt, about 3 cSt to 9 cSt, about 4 cSt to 5 cSt, about 4 cSt to 6 cSt, about 4 cSt to 7 cSt, about 4 cSt to 8 cSt, and about 4 cSt to 9 cSt.

In addition, the lube base oil of the present disclosure may have a viscosity index (VI) within a predetermined range. In an embodiment of the present disclosure, the lube base oil may have a viscosity index of about 90 or higher. For example, the viscosity index may be in a range of about 90 to 200, such as about 90 to 150, about 90 to 160, about 90 to 170, about 90 to 180, about 90 to 190, about 100 to 160, about 100 to 170, about 100 to 180, about 100 to 190, about 110 to 170, about 110 to 180, and about 110 to 190.

In addition, the lube base oil of the present disclosure may have a pour point within a predetermined range. In an embodiment of the present disclosure, the lube base oil may have a pour point of about 0° C. or lower. The lube base oil has a pour point of lower than about 0° C. For example, the lube base oil of the present disclosure may have a pour point in a range of about −60° C. to 0° C., such as about −3° C. or lower, about −10° C. or lower, about −15° C. or lower, about −20° C. or lower, about −25° C. or lower, about −30° C. or lower, about −35° C. or lower, about −40° C. or lower, about −45° C. or lower, about −50° C. or lower, and about −55° C. or lower.

The lube base oil of the present disclosure has excellent friction reduction performance and eco-friendly biodegradability, and thus can be applied to a variety of lubricating oil products.

### Lubricant Composition

The present disclosure provides a lubricant composition comprising the compound of the first aspect described above. The lubricant composition may comprise the compound serving as a lube base oil. In addition, the lubricant composition may comprise the compound serving as an additive. Hence, the lubricant composition of the present disclosure may further comprise a lube base oil different from the lube base oil described above. The lube base oil, a known lube base oil, different from the lube base oil of the present disclosure is not particularly limited, and may comprise both mineral base oils and synthetic lube base oils.

In addition, known additives used in the lubricant composition may be contained as needed. Examples of the known additives may comprise antioxidants, corrosion inhibitors, dispersants, metal detergents, anti-wear agents, extreme pressure additives, anti-foaming agents, pour point depressants, viscosity index improvers, friction modifiers, emulsifiers, viscosity modifiers, and the like, but are not limited thereto.

In an embodiment of the present disclosure, the lubricant composition may comprise the compound in an amount of less than about 50 wt % based on the total weight of the lubricant composition. Specifically, the lubricant composition may comprise the compound in an amount of greater than 0 wt % and less than about 50 wt %. For example, the lubricant composition may comprise the compound in an amount in a range of about 0.01 wt % to less than about 50 wt %, such as about 0.1 wt % to less than about 50 wt %, about 1 wt % to less than about 50 wt %, about 2 wt % to less than about 50 wt %, about 3 wt % to less than about 50 wt %, about 4 wt % to less than about 50 wt %, about 5 wt % to less than about 50 wt %, about 6 wt % to less than about 50 wt %, about 7 wt % to less than about 50 wt %, about 8 wt % to less than about 50 wt %, about 9 wt % to less than about 50 wt %, about 10 wt % to less than about 50 wt %, about 15 wt % to less than about 50 wt %, about 20 wt % to less than about 50 wt %, about 30 wt % to less than about 50 wt %, and about 40 wt % to less than about 50 wt %.

The lubricant composition of the present disclosure may have a traction coefficient equal to or less than that of the lube base oil described above. Specifically, in an embodiment of the present disclosure, the lubricant composition may have a traction coefficient of about 0.04 or less when being measured under conditions: a load of 38 N, a temperature of 100° C., a speed of 1000 mm/s, and a slide-to-roll ratio (SRR) of 100%. In an embodiment the traction coefficient may be about 0.037 or less, and in another embodiment the traction coefficient may be about 0.035 or less.

In an embodiment of the present disclosure, the lubricant composition may have a flash point of about 200° C. or higher. For example, the lubricant composition may have a flash point in a range of about 200° C. to 300° C., such as about 200° C. to 210° C., about 200° C. to 220° C., about 200° C. to 230° C., about 200° C. to 240° C., about 200° C. to 250° C., about 200° C. to 260° C., about 200° C. to 270° C., about 200° C. to 280° C., and about 200° C. to 290° C.

In an embodiment of the present disclosure, the lubricant composition may have a kinematic viscosity at a temperature of 100° C. in a range of about 2 cSt to 10 cSt, such as about 2 cSt to 3 cSt, about 2 cSt to 4 cSt, about 2 cSt to 5 cSt, about 2 cSt to 6 cSt, about 2 cSt to 7 cSt, about 2 cSt to 8 cSt, about 2 cSt to 9 cSt, about 3 cSt to 4 cSt, about 3 cSt to 5 cSt, about 3 cSt to 6 cSt, about 3 cSt to 7 cSt, about 3 cSt to 8 cSt, about 3 cSt to 9 cSt, about 4 cSt to 5 cSt, about 4 cSt to 6 cSt, about 4 cSt to 7 cSt, about 4 cSt to 8 cSt, and about 4 cSt to 9 cSt.

In addition, in an embodiment of the present disclosure, the lubricant composition may have a viscosity index of about 90 or higher. For example, the viscosity index may be in a range of about 90 to 200, such as about 90 to 150, about 90 to 160, about 90 to 170, about 90 to 180, about 90 to 190, about 100 to 160, about 100 to 170, about 100 to 180, about 100 to 190, about 110 to 170, about 110 to 180, and about 110 to 190.

Furthermore, in an embodiment of the present disclosure, the lubricant composition may have a pour point of about 0° C. or lower. In an embodiment, the lubricant composition has a pour point of lower than about 0° C. For example, the lubricant composition of the present disclosure may have a pour point in a range of about −60° C. to 0° C., such as about −3° C. or lower, about −10° C. or lower, about −15° C. or lower, about −20° C. or lower, about −25° C. or lower, about −30° C. or lower, about −35° C. or lower, about −40° C. or lower, about −45° C. or lower, about −50° C. or lower, and about −55° C. or lower.

As described above, the compound of the present disclosure may be added to the lubricant composition to improve the friction reduction performance of the entire composition as well as the biodegradation performance thereof.

Hereinafter, embodiments will be presented to aid understanding of the present disclosure. However, the following examples are provided to more easily understand the present disclosure, and the present disclosure is not limited thereto.

### EXAMPLE

### 1. Preparation of Compound

### (1) Preparation of Dihydrojasmonic Acid

200 g of methyl dihydrojasmonate and 200 mL of distilled water were added to a reaction vessel and then stirred at room temperature. Next, 53 g of sodium hydroxide was added to the reaction vessel in which the reactants were stirred, followed by heating the reaction mixture to a temperature of 80° C. and stirring the same for 12 hours. After completion of the reaction, the reaction mixture was cooled to a temperature of 0° C., and a 35% aqueous solution of hydrogen chloride was added, thereby adjusting the pH of the reaction mixture to 2. Then, 400 mL of dichloromethane was added to the reaction mixture at room temperature, and the reaction mixture was sufficiently stirred, followed by extracting an organic solvent layer. Next, magnesium sulfate was added to the organic solvent layer with stirring, filtering was performed, and the solvent was then concentrated to obtain 185 g of orange dihydrojasmonic acid.

¹H NMR (500 MHz, CDCl₃) δ 11.10-9.20 (br, 1H), 2.81-2.68 (m, 1H), 2.48-2.28 (m, 4H), 2.18-2.11 (m, 1H), 1.84-1.80 (m, 1H), 1.64-1.20 (m, 9H), 0.89-0.86 (t, 3H).

### (2) Example 1

120 g of the dihydrojasmonic acid obtained the above, 130 g of 1-hexadecanol, 360 mL of toluene, and 1.1 g of p-toluenesulfonic acid were sequentially added to a reaction vessel. Then, the reaction mixture was heated to a temperature of 140° C. with stirring for 12 hours. After completion of the reaction, 400 mL of a saturated aqueous solution of sodium hydrogen carbonate was added to the reaction vessel with stirring for 15 min at room temperature to extract an organic solvent layer. Then, magnesium sulfate was added to the organic solvent layer with stirring, filtering was performed, and the solvent was concentrated to obtain 228 g of yellow hexadecanyl dihydrojasmonate ester.

¹H NMR (500 MHz, CDCl₃) δ 4.09 (t, J=6.8 Hz, 2H), 2.83-2.58 (m, 1H), 2.37-2.20 (m, 4H), 2.14-2.11 (m, 1H), 1.81-1.77 (m, 1H), 1.79-1.49 (m, 5H), 1.41-1.26 (m, 32H), 0.88-0.86 (m, 6H).

### (3) Example 2

199 g of dodecanyl dihydrojasmonate ester was obtained in the same manner as in Example 1, except that 100 g of 1-dodecanol was added to the reaction vessel instead of 130 g of 1-hexadecanol.

¹H NMR (500 MHz, CDCl₃) δ 4.09 (t, J=6.8 Hz, 2H), 2.81-2.59 (m, 1H), 2.37-2.20 (m, 4H), 2.14-2.11 (m, 1H), 1.80-1.78 (m, 1H), 1.79-1.49 (m, 5H), 1.41-1.26 (m, 24H), 0.89-0.86 (m, 6H).

### (4) Example 3

192 g of undecanyl dihydrojasmonate ester was obtained in the same manner as in Example 1, except that 93 g of 1-undecanol was added to the reaction vessel instead of 130 g of 1-hexadecanol.

¹H NMR (500 MHz, CDCl₃) δ 4.09 (t, J=6.8 Hz, 2H), 2.82-2.59 (m, 1H), 2.37-2.20 (m, 4H), 2.14-2.11 (m, 1H), 1.80-1.78 (m, 1H), 1.79-1.49 (m, 5H), 1.41-1.26 (m, 22H), 0.89-0.86 (m, 6H).

### (5) Example 4

184 g of decanyl dihydrojasmonate ester was obtained in the same manner as in Example 1, except that 85 g of 1-decanol was added to the reaction vessel instead of 130 g of 1-hexadecanol.

¹H NMR (500 MHz, CDCl₃) δ 4.09 (t, J=6.8 Hz, 2H), 2.81-2.59 (m, 1H), 2.37-2.20 (m, 4H), 2.14-2.11 (m, 1H), 1.80-1.78 (m, 1H), 1.79-1.49 (m, 5H), 1.41-1.26 (m, 20H), 0.89-0.86 (m, 6H).

### (6) Example 5

177 g of nonanyl dihydrojasmonate ester was obtained in the same manner as in Example 1, except that 77 g of 1-nonanol was added to the reaction vessel instead of 130 g of 1-hexadecanol.

¹H NMR (500 MHz, CDCl₃) δ 4.08 (t, J=6.8 Hz, 2H), 2.81-2.58 (m, 1H), 2.39-2.20 (m, 4H), 2.16-2.06 (m, 1H), 1.80-1.76 (m, 1H), 1.65-1.47 (m, 5H), 1.41-1.26 (m, 18H), 0.88-0.86 (m, 6H).

### (7) Comparative Example 1

Methyl dihydrojasmonate with a purity of 96% or more was purchased from Sigma Aldrich for use.

### (8) Comparative Example 2

148 g of pentanyl dihydrojasmonate ester was obtained in the same manner as in Example 1, except that 47 g of 1-pentanol was added to the reaction vessel instead of 130 g of 1-hexadecanol.

¹H NMR (500 MHz, CDCl₃) δ 4.09 (t, J=6.8 Hz, 2H), 2.84-2.58 (m, 1H), 2.37-2.20 (m, 4H), 2.16-2.08 (m, 1H), 1.81-1.77 (m, 1H), 1.67-1.47 (m, 5H), 1.42-1.24 (m, 10H), 0.92-0.85 (m, 6H).

Table 2 shows the types of alcohol used in the esterification reaction with dihydrojasmonic acid, whereby each of the esters, obtained in Examples 1 to 5 and Comparative Examples 1 and 2, was produced.

TABLE 2

| Example | Alcohol |
|---|---|
| Example 1 | 1-Hexadecanol |
| Example 2 | 1-Dodecanol |
| Example 3 | 1-Undecanol |
| Example 4 | 1-Decanol |
| Example 5 | 1-Nonanol |
| Comparative | Methanol |
| Example 1 | |
| Comparative | 1-Pentanol |
| Example 2 | |

### 2. Measurement of Traction Coefficient and Analysis of Properties

The traction coefficients of the Examples and Comparative Examples were measured, and properties, such as kinematic viscosity, viscosity index, pour point, and flash point, were analyzed. Measurement conditions and measurement result values are as follows.

TABLE 3

| | | Kinematic | | | |
|---|---|---|---|---|---|
| | Traction | Viscosity (at | | | |
| Example | Coefficient | 100° C., cSt) | Viscosity Index | Pour Point (° C.) | Flash Point (° C.) |
| Example 1 | 0.0242 | 6.2 | 131 | −3 | 252 |
| Example 2 | 0.0281 | 4.6 | 119 | −45 | 250 |
| Example 3 | 0.0335 | 3.9 | 108 | −60 | 222 |
| Example 4 | 0.0345 | 3.6 | 101 | −57 | 226 |
| Example 5 | 0.0303 | 3.4 | 95 | −57 | 216 |
| Comparative | 0.0413 | 1.7 | — | −45 | 162 |
| Example 1 | | | | | |
| Comparative | 0.0417 | 2.5 | 51 | -60 | 190 |
| Example 2 | | | | | |

The traction coefficient measurement was performed using Mini Traction Machine (MTM) purchased from PCS Instruments Ltd. The traction coefficient values were measured by varying slide-to-roll ratios (SRRs) from 0% to 100% under conditions: a load of 38 N, a temperature of 100° C., and a speed of 1000 mm/s. Then, the traction coefficient values at the SRR of 100% were specified.

The kinematic viscosity measurement was performed using Kinematic Viscometer purchased from CANNON, and evaluated according to ASTM D445 test method.

The viscosity index calculation was performed based on ASTM D2270 test method using the kinematic viscosities at temperatures of 40° C. and 100° C. measured with Kinematic Viscometer purchased from CANNON.

The pour point measurement was performed using Automatic Pour/Cloud Point Testers purchased from TANAKA, and evaluated according to ASTM D6749 test method.

The flash point measurement was performed using Open Cup Flash Point Analyzers purchased from ISL, and evaluated according to ASTM D92 test method.

As seen from Table 3, it was confirmed that with the use of the alcohols having 9 or more carbon atoms, a traction coefficient of 0.04 or less was achievable under the conditions: the load of 38 N, the temperature of 100° C., the speed of 1000 mm/s, and the SRR of 100%. Thus, the compounds of Examples 1 to 5 can be used as components constituting lube base oils or lubricant compositions having a further excellent friction reduction performance.

### 3. Evaluation of Biodegradability

The biodegradability of Example 2 was evaluated. The results thereof are shown in Table 4 below.

TABLE 4

| Example | Biodegradability (%) |
|---|---|
| Example 2 | 68 |

The biodegradability evaluation was performed according to OECD 301F test method.

The biodegradability of Example 2 was 68%. Based on the OECD 301F test method, a biodegradability of 60% or higher is defined as being easily biodegradable. Thus, the compounds were confirmed to be excellently biodegradable.

While the present disclosure has been described with respect to the specific embodiments, it will be apparent to those skilled in the art that various changes and modifications of the present disclosure may be made without departing from the scope of the invention.

What is claimed is:

1. A compound represented by Formula 1, 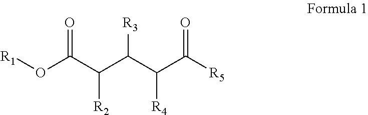
wherein R₁ is an alkyl group having 9 to 30 carbon atoms, an alkenyl group having 9 to 30 carbon atoms, or an alkynyl group having 9 to 30 carbon atoms,
R₂ is hydrogen, an alkyl group having 1 to 10 carbon atoms, an alkenyl group having 2 to 10 carbon atoms, or an alkynyl group having 2 to 10 carbon atoms, and
R₃ to R₅ are each independently hydrogen, an alkyl group having 1 to 10 carbon atoms, an alkenyl group having 2 to 10 carbon atoms, or an alkynyl group having 2 to 10 carbon atoms, or at least two of R₃ to R₅ are bonded together to form a hydrocarbon ring having 5 or 6 carbon atoms.

2. The compound of claim 1, wherein R₁ is the alkyl group having 9 to 30 carbon atoms,
R₂ is hydrogen or the alkyl group having 1 to 10 carbon atoms, and
at least two of R₃ to R₅ are bonded together to form the hydrocarbon ring having 5 or 6 carbon atoms.

3. The compound of claim 1, wherein the compound is represented by Formula 2 when R₃ and R₅ in the compound of Formula 1 are bonded together to form a hydrocarbon ring having 5 carbon atoms, 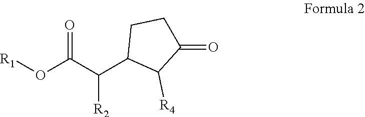
wherein R₁ is the alkyl group having 9 to 30 carbon atoms, the alkenyl group having 9 to 30 carbon atoms, or the alkynyl group having 9 to 30 carbon atoms,
R₂ is hydrogen, the alkyl group having 1 to 10 carbon atoms, the alkenyl group having 2 to 10 carbon atoms, or the alkynyl group having 2 to 10 carbon atoms, and
R₄ is hydrogen, the alkyl group having 1 to 10 carbon atoms, the alkenyl group having 2 to 10 carbon atoms, or the alkynyl group having 2 to 10 carbon atoms.

4. The compound of claim 3, wherein R₁ is the alkyl group having 9 to 30 carbon atoms,
R₂ is hydrogen or the alkyl group having 1 to 10 carbon atoms, and
R₄ is hydrogen or the alkyl group having 1 to 10 carbon atoms.

5. A lube base oil comprising the compound of claim 1.

6. The lube base oil of claim 5, wherein the lube base oil has a traction coefficient of 0.04 or less when being measured under conditions of a load of 38 N, a temperature of 100° C., a speed of 1000 mm/s, and a slide-to-roll ratio (SRR) of 100%.

7. The lube base oil of claim 5, wherein the lube base oil has a flash point of 200° C. or higher.

8. The lube base oil of claim 5, wherein the amount of the compound in the lube base oil is at least 50 wt % based on the total weight of the lube base oil.

9. A lubricant composition comprising the compound of claim 1.

10. The lubricant composition of claim 9, the lubricant composition comprises greater than 0 wt % and less than 50 wt % of the compound based on the total weight of the lubricant composition.
